(19) United States
(12) Patent Application Publication
(10) Pub. No.: US 2025/0161831 A1
(43) Pub. Date: May 22, 2025

(54) CHILDREN'S TOY

(71) Applicant: The Calm Caterpillar LLC, Cincinnati, OH (US)

(72) Inventors: Sarah Habib, Cincinnati, OH (US); Gregory J. Autore, Miamisburg, OH (US)

(21) Appl. No.: 18/512,797

(22) Filed: Nov. 17, 2023

(51) Int. Cl. A63H 3/04; A61M 21/00; A61M 21/02

(52) U.S. Cl. CPC A63H 3/04; A61M 21/02; A61M 2021/0022; A61M 2205/59

(57) ABSTRACT

A children's toy is disclosed herein. The children's toy includes a shell defining a hollow interior space and a filling housed by the shell, the filling situated within the hollow interior space. The children's toy also includes a band extending through the hollow interior space, the band coupled at a first end to a first interior surface of the shell, and the band coupled at a second end to a second interior surface of the shell. The shell and the band are configured to stretch from a contracted state to an expanded state in at least a first direction, the first direction being generally parallel with a direction in which the band longitudinally extends through the hollow interior space in the contracted state, a length of the shell is less than a length of the shell in the expanded state, and the band is biased to the contracted state and the band biasing the shell to the contracted state.

### FIELD OF THE INVENTION

The invention relates to children's toy. Specifically, the invention relates to a children's toy for breathing exercises, education, and emotional control.

### BACKGROUND OF THE INVENTION

Children, particularly between the ages of 2-6 are quickly developing people with quickly developing emotions. Furthermore, children in this age range may tantrum, become unruly, or anxious and benefit from calming down. In addition, children at these ages are at an age where their minds are absorbing educational information rapidly. Accordingly, there is a need to satisfy young children's emotional and educational development in a way that children would enjoy, as well as assist them in working through emotional turmoil and calm down.

### SUMMARY OF THE INVENTION

To address the issues related to children's emotional and educational development, a toy that is attractive and includes features that aid in children's emotional and educational development is disclosed.

In an embodiment, the children's toy includes a children's toy including a shell defining a hollow interior space is provided. The children's toy further includes a filling housed by the shell, the filling situated within the hollow interior space. The children's toy also includes a band extending through the hollow interior space, the band coupled at a first end to a first interior surface of the shell, and the band coupled at a second end to a second interior surface of the shell. The shell and the band are configured to stretch from a contracted state to an expanded state in at least a first direction, the first direction being generally parallel with a direction in which the band longitudinally extends through the hollow interior space. In the contracted state, a length of the shell is less than a length of the shell in the expanded state. The band is biased to the contracted state and the band biasing the shell to the contracted state.

In a related embodiment, the shell includes a material selected from the group consisting of long pile polyester, short pile polyester, velvet, velboa, shag, animal fur, artificial fur, cotton, silk, wool, bamboo, nylon, rayon, spandex, acrylic, and combinations thereof.

In a related embodiment, the filling includes a material selected from the group consisting of polyester, polyethylene, polypropylene, cotton, bamboo, wool, polyethylene foam, polyurethane foam, polystyrene foam, and combinations thereof long pile polyester, short pile polyester, cotton, acrylic, wool, and combinations thereof.

In a related embodiment, the band includes an elastic material.

In a related embodiment, the elastic material is flat braid elastic or round elastic cord.

In a related embodiment, the filling is configured to stretch along with the band and the shell from the contracted state to the expanded state.

In a related embodiment, the first end is mechanically coupled to the first interior surface.

In a related embodiment, the second end is mechanically coupled to the second interior surface.

In a related embodiment, the first end is chemically coupled to the first interior surface.

In a related embodiment, the second end is chemically coupled to the second interior surface.

In a related embodiment, the first end is heat-bonded to the first interior surface.

In a related embodiment, the shell comprises a plurality of lobes ordered longitudinally along the shell.

In a related embodiment, the first interior surface is located at a first longitudinal end of the shell.

In a related embodiment, the second interior surface is located at a second longitudinal end of the shell, the second longitudinal end being opposite the first longitudinal end.

In a related embodiment, the shell comprises a frontal lobe, a rear lobe, and a plurality of generally spherical segments, the generally spherical segments coupling the frontal lobe to the rear lobe.

In a related embodiment, the frontal lobe and the rear lobe maintain their shape when the children's toy transitions from the contracted state to the expanded state.

In a related embodiment, the frontal lobe comprises an appendage extending from the frontal lobe.

In a related embodiment, the appendage comprises a stem coupled to the frontal lobe at a first end of the appendage, and a bulb situated at a second end of the appendage, the second end of the appendage opposite the first end of the appendage.

In a related embodiment, the bulb comprises a diameter greater than a diameter of the stem.

In a related embodiment, each generally spherical segment is mechanically coupled to an adjacent segment at a seam.

### BRIEF DESCRIPTION OF THE DRAWINGS

The accompanying drawings, which are incorporated in and constitute a part of this specification, illustrate various embodiments of the invention and, together with the general description of the invention given above, and the detailed description of the embodiments given below, serve to explain the embodiments of the invention.

FIG. 1 is side-view of an exemplary children's toy in a contracted state.

FIG. 2 is cross-sectional view of an exemplary toy in a contracted state.

FIG. 3 is a cross-sectional view of an exemplary toy in an expanded state.

It should be understood that the appended drawings are not necessarily to scale, and may present a somewhat simplified representation of various features illustrative of the basic principles of the invention. The specific design features of the sequence of operations disclosed herein, including, for example, specific dimensions, orientations, locations, and shapes of various illustrated components, may be determined in part by the particular intended application and use environment. Certain features of the illustrated embodiments may have been enlarged or distorted relative to others to facilitate visualization and a clear understanding. In particular, thin features may be thickened, for example, for clarity or illustration.

### DESCRIPTION OF THE INVENTION

To address the issues related to children's emotional and educational development, a toy that is attractive and includes features that aid in children's emotional and educational development is disclosed. The children's toy is expandable from a first, contracted state, to a second, expanded state, by pulling longitudinally on the shell of the children's toy.

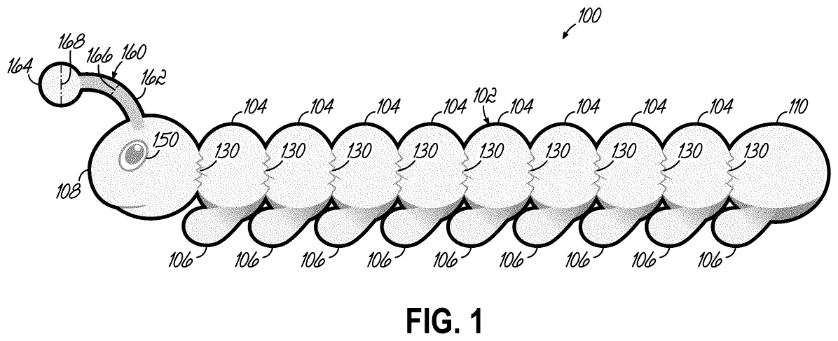

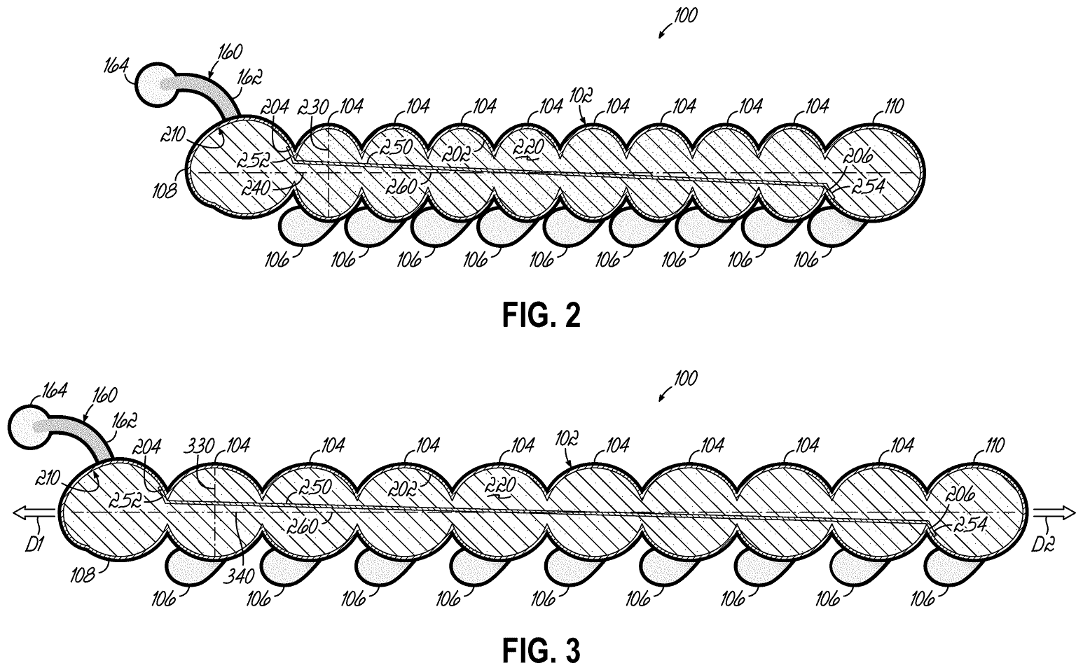

In reference to FIG. 1, a children's toy 100 is shown. The children's toy 100 includes a shell 102 which includes a frontal lobe 108, individual segments 104, and a rear lobe 110. The shell 102 defines an interior hollow space 210. Each individual segment 104 is coupled to a sequential segment 104 at longitudinal ends of each segment 104 at a seam 130. Likewise, the frontal lobe 108 is coupled to a segment 104 at a seam 130. At the seam 130 may be thread, adhesive, heat seal, or other material that couples each segment 104, frontal lobe 108, or rear lobe 110 to adjacent segments 104. Each segment 104 is a hollow casing having an interior space (shown in FIGS. 2 and 3). The interior spaces of each segment 104, the frontal lobe 108, and the rear lobe 110, together form the interior hollow space 210 defined by the shell 102 that houses a filling 220 (shown in FIGS. 2 and 3). In some embodiments, the shell 102 is made of a stretchable fabric that may be synthetic or natural. In some embodiments, the shell 102 is made of a synthetic material such as, but not limited to, long pile polyester, short pile polyester, velvet, velboa, shag, artificial/faux fur, nylon, rayon, spandex, acrylic, or combinations thereof. In some embodiments, the shell 102 is made of a natural material such as, but not limited to, cotton, silk, wool, bamboo, or combinations thereof. In some embodiments, the shell 102 is made of a combination of natural and synthetic materials, for example those previously described, and combinations thereof. In some embodiments, the shell 102 is made of pile or faux fur materials such as velvet, velboa, faux fur, or combinations thereof.

The shell 102 may be artificially or naturally scented with scents pleasant to a child to increase a child's enjoyment of the children's toy 100. The frontal lobe 108 included on one longitudinal end of the children's toy 100 may include features that add to the visual enjoyment of the children's toy 100. The frontal lobe 108 may include a face of an animal, for example a cartoon-like caterpillar as shown in FIG. 1. In such an example, the face of the animal may include eyes 150 which may be coupled to the frontal lobe 108 mechanically (such as by stitching, a tack, or other mechanical device), chemically (such as by an adhesive), or be an integral part of the frontal lobe 108. The face of the animal may also include additional features, such as appendage 160. The appendage 160 may extend from the shell 102 and include a stem 162 and a bulb 164. As shown in FIG. 1, the appendage 160 is an antennae, though any accessory feature may be used, such as an earring, hair, or other feature. The appendage 160 is easily clutched by a person using the toy 100, and can be pulled in a generally longitudinal direction to assist with expanding the toy 100 from the contracted state (shown in FIGS. 1 and 2) to the extended state (shown in FIG. 3). The bulb 164 may be located at a distal end of the appendage 160, coupled to the shell 102 via the stem 162. The bulb 164 may be a generally spherical shape, and the bulb 164 may include a diameter 168 greater than a diameter 166 of the stem 162. In embodiments where the diameter 168 of the bulb 164 is greater than the diameter 166 of the stem 162, the bulb 164 is easily clutched by children using the children's toy 100, allowing a convenient point at which to pull the children's toy 100 from the contracted state to the expanded state.

The children's toy 100 may be any shape, though shapes of friendly animals are preferred as these shapes have been shown to be pleasing to children using the children's toy 100. Of these animal, any animal may be used, and a caterpillar is shown as a non-limiting, exemplary embodiment in FIGS. 1-3. Furthermore, though only a single eye 150 or appendage 160 is shown in FIGS. 1-3, it is understood that these features may be duplicated to form symmetrical or unsymmetrical faces or multiplied to form the appearance of real animals (such as a spider's face) or imaginary animals (such as a unicorn's or dragon's face).

In some embodiments, the children's toy includes a plurality of feet 106. Similar to the appendage 160, the feet 106 provide another feature that a child may clutch to and manipulate the children's toy 100. One or more feet 106 may be coupled, either mechanically or chemically, to each segment 104, to the frontal lobe 108 (not shown), or to the rear lobe 110. The feet 106 are extensions of the shell 102 and may house filling of the same or different material as the filling 220 housed in the hollow interior space 210 by the shell 102. The feet 106 are may add alternative locations at which children using the children's toy 100 to pull and manipulate the children's toy 100.

Referring to FIG. 2, a cross-sectional view of the children's toy 100 is shown in a contracted state. While in the contracted state, a transverse diameter 230 of each individual segment 104 is longer than the corresponding longitudinal diameter 240 of each segment 104. The cross-sectional view shown in FIG. 2 includes an interior surface 202 which is coupled to a band 250. Particularly, a first end 252 of the band 250 is coupled to a first interior surface 204 of the shell 102, and a second end 254 of the band 250 is coupled to a second interior surface 206 of the shell 102. In an embodiment, the first interior surface 204 is located on the inner surface side of the frontal lobe 108 and the second interior surface 206 is located on the inner surface side of the rear lobe 110. The first end 252 of the band 250 may be coupled to the first interior surface 204 mechanically, such as by stitching, a tack, or other mechanical device, chemically, such as by a chemical adhesive, or by heat-bonding. Similarly, the second end 254 of the band 250 may be coupled to the second interior surface 206 mechanically, such as by stitching, a tack, or other mechanical device, chemically, such as by a chemical adhesive, or by heat-bonding. The band extends from the first interior surface 204 to the second interior surface 206, through the hollow interior space 210 in a direction generally parallel with a longitudinal axis 260 of the children's toy 100. In an embodiment of the children's toy 100, the first end 252 of the band 250 and the second end 254 of the band 250 are positioned on opposite sides of the longitudinal axis 260 of the children's toy 100. The band 250 is biased toward the contracted state and applies a force on the frontal lobe 108 and the rear lobe 110 as determined by Hooke's Law. When the tensile force in the band 250 is greater than the force applied in directions D1, D2, the children's toy will tend toward the contracted state. In some embodiments, the band 250 is made of a material including, but not limited to, flat braid elastic, round elastic cord, or a combination thereof.

The band 250 provides several advantages over children's toys without such the band 250. First, children's toys without the band 250, even if constructed with stretchy fabric, will stretch and may not conform back to their original shape after the stretching force is removed or reduced. This problem is overcome by the band 250 constantly applying a biasing force encouraging the children's toy 100 to the contracted state. Second, toys based on stretchy fabrics do not stretch much, however, the band 250 in the hollow interior space 220 allows for more stretch and return in the shell 102. In some embodiments, however, the shell 102 is very wrinkled and deformed when it relaxes. To prevent or reduce wrinkling, the band 250 may be placed generally down a longitudinal axis 260 down the center of the children's toy 100. Due to the segmented nature of the children's toy 100 (i.e. the shell 102 including segments 104), and the band 250 being positioned generally down the longitudinal axis down the center of the children's toy 100, a child using the children's toy 100 is able to manipulate and pull the children's toy 100 in any direction, and once the stretching force is removed, the children's toy 100 will return to the contracted state.

In some embodiments, the filling 220 may be made of a synthetic fiber such as, but not limited to, polyester, polyethylene, polypropylene, or combinations thereof. In some embodiments, the filling 220 may be made of a natural fiber such as, but not limited to, cotton, bamboo, wool, or combinations thereof. In some embodiments, the filling 220 may be made of a foam material foam materials such as but not limited to polyethylene foam, polyurethane foam, polystyrene foam, or combinations thereof. Alternatively or in addition, the filling 220 may be made of, but not limited to, any combination of these listed materials. The filling 220 material is, itself, stretchable and compactable, which gives an added advantage to the children's toy 100. Specifically, at least because the filling 220 material is stretchable and compactable, the children's toy 100 is encouraged to return to the contracted state after the force stretching the children's toy 100 is reduced or removed.

Referring to FIG. 3, a cross-sectional view of the children's toy 100 is shown in an expanded state. Transition from the contracted state to the expanded state is achieved by applying a force in opposite directions D1, D2 along the longitudinal axis 260 of the children's toy 100. Due to the stretchable nature of the children's toy 100 and the band 250, each segment 104 flattens transversely as it is stretched, resulting in the transverse diameter 330 of each individual segment 104 in the expanded state being less than the transverse diameter 230 of each segment 104 in the contracted state. Simultaneously, the longitudinal diameter 340 of each segment 104 lengthens in accordance with the force applied in the directions D1, D2, resulting in the longitudinal diameter 340 of each individual segment 104 in the expanded state being less than the longitudinal diameter 240 of each segment 104 in the contracted state. In an embodiment, the frontal lobe 108 and/or the rear lobe 110 maintain the length of their respective transverse diameter and longitudinal diameter even when the children's toy 100 is stretched from the contracted state to the expanded state.

In some embodiments, when the longitudinal force applied to the children's toy 100 to form the expanded state is removed or reduced, the band 250 applies sufficient force to return the children's toy 100 from the expanded state to the contracted state, resulting in the segments 104 each returning to their shape as they were in the original contracted state. Alternatively, the band 250 applies sufficient force to return the children's toy 100 to a second contracted state, different from the first contracted state. This second contracted state may, for example, have different lengths (i.e. longer or shorter) for the transverse diameter and the longitudinal diameter of each segment 104 than the original transverse diameter 230 and the original longitudinal diameter 240 of the segments 104 in the original contracted state.

While the present disclosure has been illustrated by the description of specific embodiments, and while the embodiments have been described in considerable detail, it is not intended that the scope of the appended claims be restricted or in any way limited to such detail. The various features discussed herein may be used alone or in any combination within and between the various embodiments. Additional advantages and modifications will readily appear to those skilled in the art. The present disclosure in its broader aspects is therefore not limited to the specific details, representative apparatus and methods and illustrative examples shown and described. Accordingly, departures may be made from such details without departing from the scope of the present disclosure.

The subject-matter of the disclosure may also relate, among others, to the following aspects:
1. A children's toy comprising:
   a shell defining a hollow interior space;
   a filling housed by the shell, the filling situated within the hollow interior space; and
   a band extending through the hollow interior space, the band coupled at a first end to a first interior surface of the shell, and the band coupled at a second end to a second interior surface of the shell,
   wherein the shell and the band are configured to stretch from a contracted state to an expanded state in at least a first direction, the first direction being generally parallel with a direction in which the band longitudinally extends through the hollow interior space,
   in the contracted state, a length of the shell is less than a length of the shell in the expanded state, and
   the band being biased to the contracted state and the band biasing the shell to the contracted state.
2. The children's toy of aspect 1, wherein the shell comprises a material selected from the group consisting of long pile polyester, short pile polyester, velvet, velboa, shag, animal fur, artificial fur, cotton, silk, wool, bamboo, nylon, rayon, spandex, acrylic, and combinations thereof.
3. The children's toy of aspect 1 or 2, wherein the filling comprises a material selected from the group consisting of polyester, polyethylene, polypropylene, cotton, bamboo, wool, polyethylene foam, polyurethane foam, polystyrene foam, and combinations thereof.
4. The children's toy of any of aspects 1-3, wherein the band comprises an elastic material.
5. The children's toy of aspect 4, wherein the elastic material is flat braid elastic or round elastic cord.
6. The children's toy of any of aspects 1-5, wherein the filling is configured to stretch along with the band and the shell from the contracted state to the expanded state.
7. The children's toy of any of aspect 1-6, wherein the first end is mechanically coupled to the first interior surface.
8. The children's toy of aspect 7, wherein the second end is mechanically coupled to the second interior surface.
9. The children's toy of any of aspects 1-8, wherein the first end is chemically coupled to the first interior surface.
10. The children's toy of aspect 9, wherein the second end is chemically coupled to the second interior surface.
11. The children's toy of any of aspects 1-10, wherein the first end is heat-bonded to the first interior surface.
12. The children's toy of any of aspects 1-11, wherein the shell comprises a plurality of lobes ordered longitudinally along the shell.
13. The children's toy of any of aspects 1-12, wherein the first interior surface is located at a first longitudinal end of the shell.
14. The children's toy of aspect 13, wherein the second interior surface is located at a second longitudinal end of the shell, the second longitudinal end being opposite the first longitudinal end.
15. The children's toy of any of aspects 1-14, wherein the shell comprises a frontal lobe, a rear lobe, and a plurality of generally spherical segments, the generally spherical segments coupling the frontal lobe to the rear lobe.
16. The children's toy of any of claims 1-15, wherein the frontal lobe and the rear lobe maintain their shape when the children's toy transitions from the contracted state to the expanded state.
17. The children's toy of any of aspects 1-16, wherein the frontal lobe comprises an appendage extending from the frontal lobe.
18. The children's toy of aspect 17, wherein the appendage comprises a stem coupled to the frontal lobe at a first end of the appendage, and a bulb situated at a second end of the appendage, the second end of the appendage opposite the first end of the appendage.
19. The children's toy of aspect 18, wherein the bulb comprises a diameter greater than a diameter of the stem.
20. The children's toy of aspect 15, wherein each generally spherical segment is mechanically coupled to an adjacent segment at a seam.

Having described the invention, what is claimed is:

1. A children's toy comprising:
a shell defining a hollow interior space;
a filling housed by the shell, the filling situated within the hollow interior space; and
a band extending through the hollow interior space, the band coupled at a first end to a first interior surface of the shell, and the band coupled at a second end to a second interior surface of the shell,
wherein the shell and the band are configured to stretch from a contracted state to an expanded state in at least a first direction, the first direction being generally parallel with a direction in which the band longitudinally extends through the hollow interior space,
in the contracted state, a length of the shell is less than a length of the shell in the expanded state, and
the band being biased to the contracted state and the band biasing the shell to the contracted state.

2. The children's toy of claim 1, wherein the shell comprises a material selected from the group consisting of long pile polyester, short pile polyester, velvet, velboa, shag, animal fur, artificial fur, cotton, silk, wool, bamboo, nylon, rayon, spandex, acrylic, and combinations thereof.

3. The children's toy of claim 1, wherein the filling comprises a material selected from the group consisting of polyester, polyethylene, polypropylene, cotton, bamboo, wool, polyethylene foam, polyurethane foam, polystyrene foam, and combinations thereof.

4. The children's toy of claim 1, wherein the band comprises an elastic material.

5. The children's toy of claim 4, wherein the elastic material is flat braid elastic or round elastic cord.

6. The children's toy of claim 1, wherein the filling is configured to stretch along with the band and the shell from the contracted state to the expanded state.

7. The children's toy of claim 1, wherein the first end is mechanically coupled to the first interior surface.

8. The children's toy of claim 7, wherein the second end is mechanically coupled to the second interior surface.

9. The children's toy of claim 1, wherein the first end is chemically coupled to the first interior surface.

10. The children's toy of claim 9, wherein the second end is chemically coupled to the second interior surface.

11. The children's toy of claim 1, wherein the first end is heat-bonded to the first interior surface.

12. The children's toy of claim 1, wherein the shell comprises a plurality of lobes ordered longitudinally along the shell.

13. The children's toy of claim 1, wherein the first interior surface is located at a first longitudinal end of the shell.

14. The children's toy of claim 13, wherein the second interior surface is located at a second longitudinal end of the shell, the second longitudinal end being opposite the first longitudinal end.

15. The children's toy of claim 1, wherein the shell comprises a frontal lobe, a rear lobe, and a plurality of generally spherical segments, the generally spherical segments coupling the frontal lobe to the rear lobe.

16. The children's toy of claim 15, wherein the frontal lobe and the rear lobe maintain their shape when the children's toy transitions from the contracted state to the expanded state.

17. The children's toy of claim 15, wherein the frontal lobe comprises an appendage extending from the frontal lobe.

18. The children's toy of claim 17, wherein the appendage comprises a stem coupled to the frontal lobe at a first end of the appendage, and a bulb situated at a second end of the appendage, the second end of the appendage opposite the first end of the appendage.

19. The children's toy of claim 18, wherein the bulb comprises a diameter greater than a diameter of the stem.

20. The children's toy of claim 15, wherein each generally spherical segment is mechanically coupled to an adjacent segment at a seam.
